(19) United States
(12) Patent Application Publication
(10) Pub. No.: US 2005/0027339 A1
(43) Pub. Date: Feb. 3, 2005

(54) System and method for providing a medical lead body

(75) Inventors: Michael P. Schrom, Wyoming Township, MN (US); Charles F. Lehman, Minneapolis, MN (US); Mark Gerald Schrom, Hugo, MN (US)

Correspondence Address: DOCKET CLERK, DM/ANSI, P.O. BOX 802432, DALLAS, TX, 75380, US

(73) Assignee: MICRONET MEDICAL, INC., White Bear Lake, MN (US)

(21) Appl. No.: 10/630,219

(22) Filed: Jul. 29, 2003

(51) Int. Cl. A61N 1/05

(52) U.S. Cl. 607/116

(57) ABSTRACT

An implantable lead and lead body, method of manufacturing the same, and a system and method for stimulating a portion of a body is disclosed. In one advantageous embodiment, a lead body assembly is formed by placing an inner layer of extrusion material on a mandrel, wrapping a plurality of conductors coated with extrusion material around the inner layer, and placing an outer layer of extrusion material over the plurality of conductors. Heat shrink tubing is placed over the lead body assembly and the lead body assembly is heated to melt the extrusion material. The melted extrusion material is compressed around the plurality of conductors. The assembly is then cooled and the heat shrink tubing is removed. The solidified extrusion material forms a protective wall that encapsulates the plurality of conductors in the lead body.

### CROSS-REFERENCE TO RELATED PATENT DOCUMENTS

The present disclosure is related to the inventions disclosed in the following United States patent applications:

United States Patent Application No. [Attorney Docket Number 03-003] filed concurrently herewith, entitled “System and Method for Providing a Medical Lead Body With Dual Conductor Layers”; and

United States Patent Application No. [Attorney Docket Number 03-009] filed concurrently herewith, entitled “System and Method for Providing A Medical Lead Body Having Conductors That Are Wound in Opposite Directions.”

These patent applications are commonly owned by the assignee of the present invention. The disclosures of the related United States patent applications are incorporated herein by reference for all purposes as if fully set forth.

### TECHNICAL FIELD OF THE INVENTION

The present invention generally relates to medical leads and, more particularly, to a system and method for manufacturing an implantable lead that includes a lead body having conductors that are located between an inner layer of extrusion material and an outer layer of extrusion material.

### BACKGROUND OF THE INVENTION

Electrical signals may be used in a variety of medical applications to provide electrical stimulation to various parts of the body of a patient. For example, electrical signals may be used to modulate the amount of pain perceived by a patient by electrically stimulating a site near one or more nerves of the patient. A source of electrical signals may be implanted within the body of a patient. Electrical signals are conducted from the source of electrical signals to the stimulation site of the patient through a lead implanted within the body of the patient.

A lead generally includes a thin, flexible, lead body that contains electrically conducting conductors (e.g., wires) that extend from a first end of the lead (the proximal end) to a second end of the lead (the distal end). The lead body includes insulating material for covering and electrically insulating the electrically conducting conductors. The proximal end of the lead further includes an electrical contact that may be coupled to a source of electrical signals and the distal end of the lead includes an electrode that may be placed at the stimulation site within the body of the patient.

A prior art manufacturing process that the inventors developed for a lead involved placing a plurality of electrically conducting conductors on a layer of extrusion material placed on an underlying mandrel. This method was developed for only up to four conductors, because the conductors ran longitudinally along the length of the mandrel. Because only four wires were used, concern about insulating the conductors were minimized by evenly spacing the wires along the length, something that was simplified because of placement of the wires along the length of the mandrel. Greater than four conductors caused concern for mass production because of narrowing spacing requirements tended to cause conductor interference and shorts, since it became more difficult to evenly space the conductors.

After the conductors were in place on the extrusion material on the mandrel in this method, the conductors were then covered with another layer of extrusion material and a heat shrink process is applied to melt the extrusion material. The extrusion material was then cooled to form a lead body that encapsulates the conductors.

Different prior art conductors suggest that the conductors may be wound around a cylindrically shaped mandrel in a spiral manner. Here, a mechanical comb is utilized in the prior art winding process to keep the conductors separated as the conductors are wound around the mandrel. The use of a mechanical comb can sometimes cause the pitch of the conductors to vary. The term “pitch” refers to the distance along the axis of the mandrel that represents one turn of conductor around the mandrel.

The use of mechanical combs can also sometimes damage the conductors. Prior art manufacturing methods can also result in a lead body that has variable (non-uniform) conductor pitches for the conductors in the lead body. Prior art manufacturing methods can also result in a lead body that has variable (non-uniform) wall thicknesses. Prior art manufacturing methods also can result in the creation of lead bodies that have relatively large diameters.

Larger electrode-carrying catheters in the prior art (such as those used in cardiology applications) may utilize electrically conducting wires that are spirally wound around a cylindrically shaped wire core. For example, U.S. Pat. No. 5,417,208 issued to Winkler describes an electrode-carrying catheter that comprises insulated wires (or non-insulated wires) that are spirally wound under hand tension around a cylindrically symmetrical wire core. The wires are embedded in a soft plastic covering (such as polyurethane having a durometer hardness of 80A available under the trade name Tecoflex) over-extruded over the wire core. The wires are embedded in the plastic covering to preclude accidental movement of the wires with respect to the wire core. Subsequently, an insulating layer of plastic is over-extruded over the soft core covering layer. This insulating layer forms a hard outer layer.

There is a need in the art for an improved system and method for manufacturing a lead body. In particular, there is a need in the art for a system and method for manufacturing a lead body that is capable of protecting and accurately placing electrically conducting conductors within the lead body during the manufacturing process. There is also a need in the art for a system and method for manufacturing a lead body that has a minimal diameter.

### SUMMARY OF THE INVENTION

The present invention is directed to a system and method for manufacturing a medical lead that includes a lead body composed of a plurality of conductors that have been previously coated with extrusion material before the plurality of conductors are assembled to form the lead body.

In one advantageous embodiment of the present invention, a lead body assembly is formed by placing an inner layer of extrusion material on a mandrel. A plurality of conductors is coated with extrusion material and each coated conductor is wrapped around the inner layer of extrusion material on the mandrel. An outer layer of extrusion material is then placed over the plurality of conductors that are coated with extrusion material. Heat shrink tubing is then placed over the lead body assembly and the lead body assembly is heated to melt the extrusion material. The melted extrusion material is compressed around the plurality of conductors as the heat shrink tubing shrinks. The lead body assembly is then cooled to form a lead body and the heat shrink tubing is removed. The solidified extrusion material forms a protective wall that encapsulates the plurality of conductors in the lead body. The lead body is then removed from the mandrel.

In another advantageous embodiment of the present invention, a lead body assembly is formed by coating a plurality of conductors with extrusion material and wrapping each of the coated conductors around a mandrel. An outer layer of extrusion material is then placed over the plurality of conductors that are coated with extrusion material. Heat shrink tubing is then placed over the lead body assembly and the lead body assembly is heated to melt the extrusion material. The melted extrusion material is compressed around the plurality of conductors as the heat shrink tubing shrinks. The lead body assembly is then cooled to form a lead body and the heat shrink tubing is removed. The solidified extrusion material forms a protective wall that encapsulates the plurality of conductors in the lead body. The lead body is then removed from the mandrel.

In another advantageous embodiment of the present invention, a lead body assembly is formed by placing an inner layer of extrusion material on a mandrel. A plurality of conductors is coated with extrusion material and each coated conductor is wrapped around the inner layer of extrusion material on the mandrel. Heat shrink tubing is then placed over the lead body assembly and the lead body assembly is heated to melt the extrusion material. The melted extrusion material is compressed around the plurality of conductors as the heat shrink tubing shrinks. The lead body assembly is then cooled to form a lead body and the heat shrink tubing is removed. The solidified extrusion material forms a protective wall that encapsulates the plurality of conductors in the lead body. The lead body is then removed from the mandrel.

In another advantageous embodiment of the present invention, a lead body assembly is formed by coating a plurality of conductors with extrusion material and wrapping each of the coated conductors around a mandrel. Heat shrink tubing is then placed over the lead body assembly and the lead body assembly is heated to melt the extrusion material. The melted extrusion material is compressed around the plurality of conductors as the heat shrink tubing shrinks. The lead body assembly is then cooled to form a lead body and the heat shrink tubing is removed. The solidified extrusion material forms a protective wall that encapsulates the plurality of conductors in the lead body. The lead body is then removed from the mandrel.

The foregoing has outlined rather broadly the features and technical advantages of the present invention so that those skilled in the art may better understand the detailed description of the invention that follows. Additional features and advantages of the invention will be described hereinafter that form the subject of the claims of the invention. Those skilled in the art should appreciate that they may readily use the conception and the specific embodiment disclosed as a basis for modifying or designing other structures for carrying out the same purposes of the present invention. Those skilled in the art should also realize that such equivalent constructions do not depart from the spirit and scope of the invention in its broadest form.

### BRIEF DESCRIPTION OF THE DRAWINGS

For a more complete understanding of the present invention, and the advantages thereof, reference is now made to the following descriptions and the accompanying drawings, wherein like numbers designate like objects, and in which:

FIG. 1 illustrates a perspective view of a lead constructed in accordance with the present invention;

FIG. 2 illustrates a lead of the present invention connected to a stimulation source including an implantable pulse generator (IPG);

FIG. 3 illustrates a lead of the present invention connected to a stimulation source including a radio frequency receiver;

FIG. 4 illustrates a cross sectional view of a first embodiment of a lead body assembly of the present invention comprising an inner layer of extrusion material, a plurality of conductors coated with a layer of extrusion material, and an outer layer of extrusion material;

FIG. 5 illustrates a cross sectional view of a first embodiment of the lead body of the present invention formed by subjecting the lead body assembly shown in FIG. 4 to melting and compression;

FIG. 6 illustrates a cross sectional view of a second embodiment of a lead body assembly of the present invention comprising a plurality of conductors coated with a layer of extrusion material and an outer layer of extrusion material;

FIG. 7 illustrates a cross sectional view of a second embodiment of the lead body of the present invention formed by subjecting the lead body assembly shown in FIG. 6 to melting and compression;

FIG. 8 illustrates a cross sectional view of a third embodiment of a lead body assembly of the present invention comprising an inner layer of extrusion material and a plurality of conductors coated with a layer of extrusion material;

FIG. 9 illustrates a cross sectional view of a third embodiment of the lead body of the present invention formed by subjecting the lead body assembly shown in FIG. 8 to melting and compression;

FIG. 10 illustrates a cross sectional view of a fourth embodiment of a lead body assembly of the present invention comprising a plurality of conductors coated with a layer of extrusion material;

FIG. 11 illustrates a cross sectional view of a fourth embodiment of the lead body of the present invention formed by subjecting the lead body assembly shown in FIG. 10 to melting and compression;

FIG. 12 illustrates a perspective side view of a mandrel showing how an exemplary conductor may be coiled around the axial length of the mandrel.

FIG. 13 illustrates a perspective side view of a mandrel showing how a plurality of conductors may be placed along the axial length of the mandrel.

FIG. 14 is a flow diagram illustrating the steps of an advantageous embodiment of a method for making a first embodiment of the lead body of the present invention;

FIG. 15 is a flow diagram illustrating the steps of an advantageous embodiment of a method for making a second embodiment of the lead body of the present invention;

FIG. 16 is a flow diagram illustrating the steps of an advantageous embodiment of a method for making a third embodiment of the lead body of the present invention; and

FIG. 17 is a flow diagram illustrating the steps of an advantageous embodiment of a method for making a fourth embodiment of the lead body of the present invention.

### DETAILED DESCRIPTION OF THE INVENTION

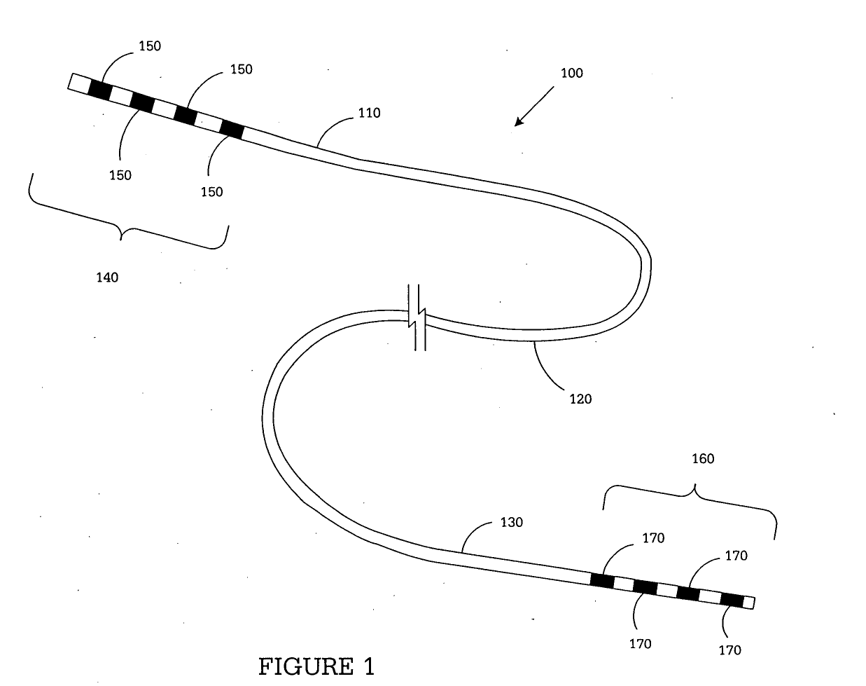

FIGS. 1 through 17, discussed below, and the various embodiments used to describe the principles of the present invention in this patent document are by way of illustration only and should not be construed in any way to limit the scope of the invention. Those skilled in the art will understand that the principles of the present invention may be implemented in any suitably modified medical lead.

FIG. 1 illustrates an advantageous embodiment of a lead 100 of the present invention. Lead 100 includes a flexible lead body 120 having a proximal end 110 and a distal end 130. Proximal end 110 of lead body 120 is coupled to an electrical contact 140. Distal end 130 of lead body 120 is coupled to electrode 160. Electrical contact 140 includes portions of lead body 120 and a plurality of contact electrodes 150 (also sometimes referred to as ring electrodes 150). Electrode 160 includes portions of lead body 120 and a plurality of band electrodes 170 (also sometimes referred to as ring electrodes 170). Although four contact electrodes 150 and four band electrodes 170 are shown in FIG. 1, it is understood that the present invention is not limited to the use of exactly four contact electrodes 150 or four band electrodes 170.

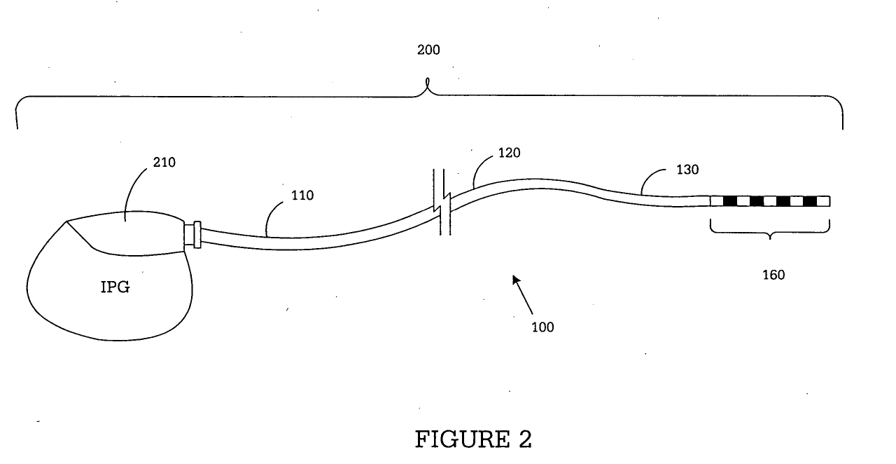

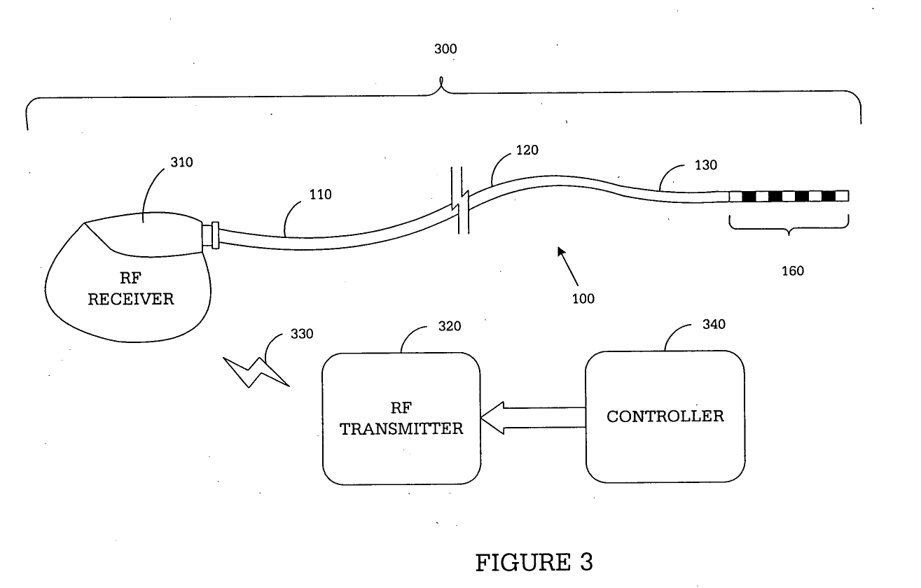

FIG. 2 and FIG. 3 illustrate different embodiments of a system (200, 300) for generating and applying a stimulus to a tissue or to a certain location of a body. In general terms, the system (200, 300) includes a stimulation or energy source (210, 310) and a lead 100 for application of the stimulus. The lead 100 shown in FIG. 2 and in FIG. 3 is the lead of the present invention.

FIG. 2 illustrates a lead 100 of the present invention connected to a stimulation source 210. The stimulation source 210 shown in FIG. 2 includes an implantable pulse generator (IPG). As is well known in the art, an implantable pulse generator (IPG) is capable of being implanted within a body (not shown) that is to receive electrical stimulation from the stimulation source 210. An exemplary implantable pulse generator (IPG) may be one manufactured by Advanced Neuromodulation Systems, Inc., such as the Genesis® System, part numbers 3604, 3608, 3609, and 3644. Reference numeral 200 refers to the system including the lead 100 and the stimulation source 210.

Electrical contact 140 is not visible in FIG. 2 because electrical contact 140 is situated within a receptacle (not shown) of stimulation source 210. Electrical contact 140 is electrically connected to a generator (not shown) of electrical signals within stimulation source 210. Stimulation source 210 generates and sends electrical signals via lead 100 to electrode 160. Electrode 160 is located at a stimulation site (not shown) within the body that is to receive electrical stimulation from the electrical signals. A stimulation site may be, for example, adjacent to one or more nerves in the central nervous system (e.g., spinal cord). The band electrodes 170 of electrode 160 conduct electrical signals from electrode 160 to the stimulation site. Stimulation source 210 is capable of controlling the electrical signals by varying signal parameters (e.g., intensity, duration, frequency) in response to control signals that are provided to stimulation source 210.

FIG. 3 illustrates a lead 100 of the present invention connected to a stimulation source 310. The stimulation source 310 shown in FIG. 3 includes a radio frequency (RF) receiver. As is well known in the art, a stimulation source 310 comprising a radio frequency (RF) receiver is capable of being implanted within the body (not shown) that is to receive electrical stimulation from the stimulation source 310. Exemplary RF receiver 310 may be those RF receivers manufactured by Advanced Neuromodulation Systems, Inc., such as the Renew® System, part numbers 3408 and 3416. Reference numeral 300 refers to the system including the lead 100 and the stimulation source 310. System 300 may also include the optional components 320 and 340 described below.

Electrical contact 140 is not visible in FIG. 3 because electrical contact 140 is situated within a receptacle (not shown) of stimulation source 310. Electrical contact 140 is electrically connected to a generator (not shown) of electrical signals within stimulation source 310. Stimulation source 310 generates and sends electrical signals via lead 100 to electrode 160. Electrode 160 is located at a stimulation site (not shown) within the body that is to receive electrical stimulation from the electrical signals. A stimulation site may be, for example, adjacent to one or more nerves in the central nervous system (e.g., spinal cord). The band electrodes 170 of electrode 160 conduct electrical signals from electrode 160 to the stimulation site. Stimulation source 310 is capable of controlling the electrical signals by varying signal parameters (e.g., intensity, duration, frequency) in response to control signals that are provided to stimulation source 310.

As shown in FIG. 3, the radio frequency (RF) receiver within stimulation source 310 is capable of receiving radio signals from a radio frequency (RF) transmitter 320. The radio signals are represented in FIG. 3 by radio link symbol 330. Radio frequency (RF) transmitter 320 and controller 340 are located outside of the body that is to receive electrical stimulation from stimulation source 310. A user of stimulation source 310 may use controller 340 to provide the control signals for the operation of stimulation source 310. Controller 340 provides the control signals to radio frequency (RF) transmitter 320. Radio frequency (RF) transmitter 320 transmits the control signals to the radio frequency (RF) receiver in stimulation source 310. Stimulation source 310 uses the control signals to vary the signal parameters of the electrical signals that are transmitted through electrical contact 140, lead body 120, and electrode 160 to the stimulation site. Exemplary RF transmitter 320 may be those RF transmitters manufactured by Advanced Neuromodulation Systems, Inc., such as the Renew® System, part numbers 3508 and 3516.

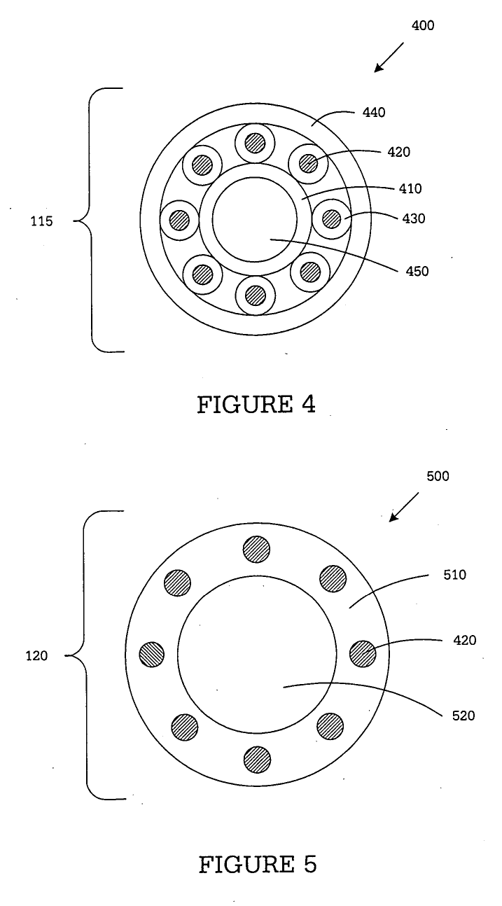

FIG. 4 illustrates a cross sectional view of a first embodiment of a lead body assembly 115 of the present invention. Lead body assembly 115 includes (1) an inner layer 410 of extrusion material, (2) a plurality of conductors 420 in which each conductor 420 is coated with a layer of extrusion material 430, and (3) an outer layer 440 of extrusion material. A lumen 450 is formed by the inner wall of inner layer 410. The portions of the first embodiment of lead body assembly 115 shown in FIG. 4 are collectively referred to with reference numeral 400.

An advantageous embodiment of a method for making the first embodiment of lead body 120 (shown in FIG. 5) will now be described. An inner layer 410 of extrusion material is placed on a cylindrically shaped mandrel (not shown). After the lead body 120 is removed from the mandrel, the space formerly occupied by the mandrel will form lumen 450 within inner layer 410. Each conductor 420 of the plurality of conductors 420 is coated with a layer 430 of the same extrusion material that is used to form inner layer 410. Alternatively, the extrusion material used to form layer 430 may not be the same type of extrusion material that is used to form inner layer 410. Each conductor 420 of the plurality of conductors 420 is cylindrically wrapped around (i.e., coiled around) the inner layer 410 of extrusion material. The layer 430 of extrusion material around each conductor 420 ensures that the conductors 420 are uniformly spaced. An outer layer 440 of extrusion material is placed over the plurality of conductors 420. The outer layer 440 of extrusion material forms an external coating over the plurality of conductors 420 as shown in FIG. 4.

In an alternative embodiment of the method of the present invention, each conductor 420 of the plurality of conductors 420 is not coiled around the inner layer 410 of extrusion material, but instead is placed lengthwise along the axial length of inner layer 410. An outer layer 440 of extrusion material is placed over the plurality of conductors 420 in the same manner as in the case of the coiled conductors 420.

The extrusion material is formed of an insulating material typically selected based upon biocompatibility, biostability and durability for the particular application. The extrusion material may be silicone, polyurethane, polyethylene, polyimide, polyvinylchloride, PTFT, EFTE, or other suitable materials known to those skilled in the art. Alloys or blends of these materials may also be formulated to control the relative flexibility, torqueability, and pushability of the lead body 120. Depending on the particular application, the diameter of the lead body 120 may be any size, though a smaller size is more desirable for neurological and myocardial mapping/ablation leads and neuromodulation and stimulation leads.

The conductors may take the form of solid conductors, drawn-filled-tube (DFT), drawn-brazed-strand (DBS), stranded conductors or cables, ribbons conductors, or other forms known or recognized to those skilled in the art. The composition of the conductors may include aluminum, stainless steel, MP35N, platinum, gold, silver, copper, vanadium, alloys, or other conductive materials or metals known to those of ordinary skill in the art. The number, size, cross-sectional shape, and composition of the conductors will depend on the particular application for the lead body 120.

As previously mentioned, the conductors may be configured along the lead body 120 in a straight orientation or cylindrically or helically wound around the lumen 450 at the center of the lead body 120. The conductors are typically insulated from the lumen 450, and from each other, and from the external surface of the lead body 120 by the extrusion material. As also previously mentioned, the extrusion material may be of single composition, or of multiple layers of the same or different materials.

In one embodiment of the invention, the combined portions 400 of lead body assembly 115 are then covered with heat shrink tubing (not shown) and heat is applied. The heat melts the layers (410, 430 and 440) of extrusion material and the melted extrusion material flows together to form an integral body. The heat shrink tubing holds and compresses the melted extrusion material around the conductors that are located within the extrusion material to create a unitary body lead 500 as shown in FIG. 5. The conductors 420 in unitary body lead 500 are contained in the unitary core, that comprises a unitary or unified wall 510, lumen 520 and conductors 420. The conductors 420 are each within the wall 510 of the unitary body lead 500 and may be centered within the unitary wall 510.

Thus, once formed as described above, there is no need to have a separate or secondary electrical insulation material (separate from the extrusion material that forms wall 510) surrounding the conductors as in the prior art. This is because the unitary construction of wall 510 acts as the electrical insulation material and forms the unitary core of the unitary body. This is true for embodiments of this invention including those described below.

Wall 510 is formed from the layers that include the layers (410, 430 and 440) of extrusion material shown in FIG. 4. As known, the various extrusion materials may be of a like kind or may be formulated using different materials such that when formed as a unitary body, the lead body will have a desired consistence, flexibility, electrically conductive properties, or other such functionality as may be desired. This holds true for all embodiments of the invention described below.

In the embodiment described above, the unitary body lead 500 is cooled and the heat shrink tubing removed. Lumen 520 is formed when the unitary body lead 500 is removed from the mandrel (not shown). There may be some release of coiled tension in the conductors 420 when the heat shrink tubing is removed. This holds true for all embodiments of the invention described below.

While the previous paragraphs describe one embodiment of forming the unitary body, those skilled in the art will recognize that other like methods may be used. For example, some of the other possible ways of forming the lead without heat shrink could be: a single hot die, successively smaller dies wherein the dies are used to draw the product to a final outside diameter. Other methods could be a compression mold or hot die drawing or other methods familiar to those in the arts. In fact, as those skilled will understand, any heating method that results in the wires becoming imbedded in a homogenous plastic or unitary body may be used. This holds true for all embodiments of the invention described below.

The present invention provides a layer 430 of extrusion material around each conductor 420. This protective layer 430 of extrusion material provides an electrical barrier between each of the conductors 420. This process also provides a significant improvement over the prior art method that uses a mechanical comb in the winders to try to keep the conductors 420 separate. The protective layer 430 of extrusion material also allows the present invention to create leads that are smaller and thinner than prior art leads.

The method of the present invention provides several advantages over prior art methods. Advantages of the method of the present invention include: (1) more accurate conductor placement during the process of coiling the conductor around a mandrel, (2) more accurate conductor pitches, (3) improved pitch consistency, (4) more conductor protection during the process of coiling the conductor around the mandrel, and (5) precise centering of the conductors within the resulting unitary body.

In addition, the apparatus and method of the present invention makes possible the construction of lead bodies that have a smaller diameter than prior art lead bodies. That is, the lead bodies of the present invention may be made thinner than prior art lead bodies. In general, the inventive lead body diameter will be smaller than 34 French and can be smaller than 9 French. (This holds true for the embodiments described below). The cylindrically symmetrical embodiment of the lead body 120 of the invention can also better withstand lateral stretching than prior art lead bodies.

The lead body assembly 115 shown in FIG. 4 has been described as having cylindrical symmetry. It is noted that other types of geometrical cross-sectional shapes (e.g., rectangular) could be used if a different shape is desired for a particular application.

The lead body assembly 115 shown in FIG. 4 and the lead body 120 shown in FIG. 5 have been shown as having eight conductors 420. The use of eight conductors 420 is merely an example. It is understood that more than eight conductors 420 may be used. It is also understood that fewer than eight conductors 420 may be used.

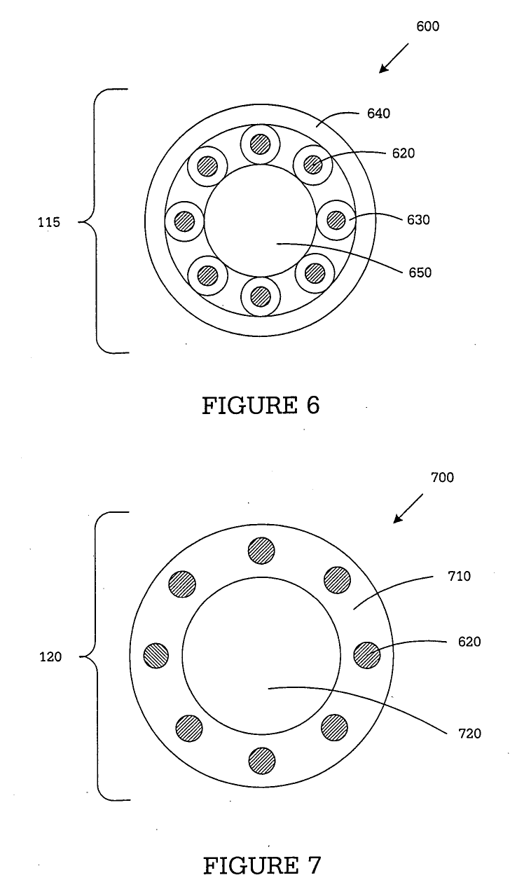

FIG. 6 illustrates a cross sectional view of a second embodiment of a lead body assembly 115 of the present invention. Lead body assembly 115 includes (1) a plurality of conductors 620 in which each conductor 620 is coated with a layer of extrusion material 630, and (2) an outer layer 640 of extrusion material. A lumen 650 is formed by the plurality of coated conductors 620. The portions of the second embodiment of lead body assembly 115 shown in FIG. 6 are collectively referred to with reference numeral 600.

An advantageous embodiment of a method for making the second embodiment of lead body 120 (shown in FIG. 7) will now be described. A plurality of conductors 620 is provided in which each conductor 620 is coated with a layer 630 of extrusion material. Each conductor 620 of the plurality of conductors 620 is cylindrically wrapped around (i.e., coiled around) a cylindrically shaped mandrel (not shown). After the lead body 120 is removed from the mandrel, the space formerly occupied by the mandrel will form lumen 650 within the plurality of coated conductors 620. The layer 630 of extrusion material around each conductor 620 ensures that the conductors 620 are uniformly spaced. An outer layer 640 of extrusion material is placed over the plurality of conductors 620. The outer layer 640 of extrusion material forms an external coating over the plurality of conductors 620 as shown in FIG. 6.

In an alternative embodiment of the method of the present invention, each conductor 620 of the plurality of conductors 620 is not coiled around the cylindrically shaped mandrel, but instead is placed lengthwise along the axial length of the cylindrically shaped mandrel. An outer layer 640 of extrusion material is placed over the plurality of conductors 620 in the same manner as in the case of the coiled conductors 620.

The combined portions 600 of lead body assembly 115 are then covered with heat shrink tubing (not shown) and heat is applied. The heat melts the layers (630 and 640) of extrusion material and the melted extrusion material flows together to form an integral body. The heat shrink tubing holds and compresses the melted extrusion material around the conductors that are located within the extrusion material to create a unitary body lead 700 as shown in FIG. 7. The conductors 620 in unitary body lead 700 may each be centered within the wall 710 of the unitary body lead 700. Wall 710 is formed from the layers that include the layers (630 and 640) of extrusion material shown in FIG. 6. The unitary body lead 700 is cooled and the heat shrink tubing removed. Lumen 720 is formed when the unitary body lead 700 is removed from the mandrel (not shown). There may be some release of coiled tension in the conductors 620 when the heat shrink tubing is removed.

The lead body assembly 115 shown in FIG. 6 has been described as having cylindrical symmetry. It is noted that other types of geometrical cross-sectional shapes (e.g., rectangular) could be used if a different shape is desired for a particular application.

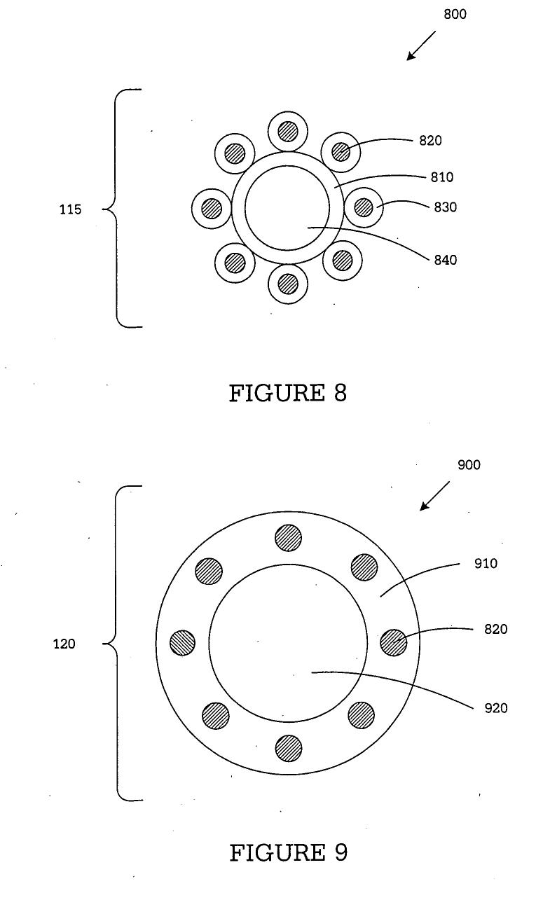

FIG. 8 illustrates a cross sectional view of a third embodiment of a lead body assembly 115 of the present invention. Lead body assembly 115 includes (1) a plurality of conductors 820 in which each conductor 820 is coated with a layer of extrusion material 830, and (2) an inner layer 810 of extrusion material. A lumen 840 is formed by the inner wall of inner layer 810. The portions of the third embodiment of lead body assembly 115 shown in FIG. 8 are collectively referred to with reference numeral 800.

An advantageous embodiment of a method for making the third embodiment of lead body 120 (shown in FIG. 9) will now be described. An inner layer 810 of extrusion material is placed on a cylindrically shaped mandrel (not shown). After the lead body 120 is removed from the mandrel, the space formerly occupied by the mandrel will form lumen 840 within inner layer 810. Each conductor 820 of a plurality of conductors 820 is coated with a layer 830 of extrusion material. Each conductor 820 of the plurality of conductors 820 is cylindrically wrapped around (i.e., coiled around) the inner layer 810 of extrusion material. The layer of extrusion material 830 around each conductor 820 ensures that the conductors 820 are uniformly spaced as shown in FIG. 8.

In an alternative embodiment of the method of the present invention, each conductor 820 of the plurality of conductors 820 is not coiled around inner layer 810 of extrusion material, but instead is placed lengthwise along the axial length of the inner layer 810 of extrusion material.

The combined portions 800 of lead body assembly 115 are then covered with heat shrink tubing (not shown) and heat is applied. The heat melts the layers (810 and 820) of extrusion material and the melted extrusion material flows together to form an integral body. The heat shrink tubing holds and compresses the extrusion material around the conductors that are located within the extrusion material to create a unitary body lead 900 as shown in FIG. 9. The conductors 820 in unitary body lead 900 may each be centered within the wall 910 of the unitary body lead 900. Wall 910 is formed from the layers that include the layers (810 and 830) of extrusion material shown in FIG. 8. The unitary body lead 900 is then cooled and the heat shrink tubing removed. Lumen 920 is formed when the unitary body lead 900 is removed from the mandrel (not shown). There may be some release of coiled tension in the conductors when the heat shrink tubing is removed.

The lead body assembly 115 shown in FIG. 8 has been described as having cylindrical symmetry. It is noted that other types of geometrical cross-sectional shapes (e.g., rectangular) could be used if a different shape is desired for a particular application.

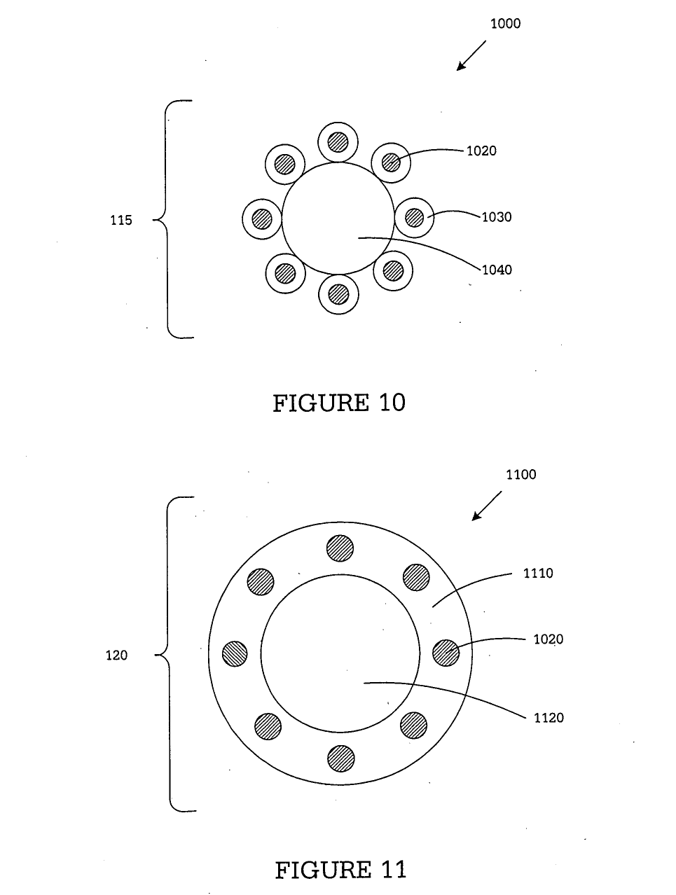

FIG. 10 illustrates a cross sectional view of a fourth embodiment of a lead body assembly 115 of the present invention. Lead body assembly 115 includes a plurality of conductors 1020 in which each conductor 1020 is coated with a layer of extrusion material 1030. A lumen 1040 is formed by the plurality of conductors 1020. The portions of the fourth embodiment of lead body assembly 115 shown in FIG. 10 are collectively referred to with reference numeral 1000.

An advantageous embodiment of a method for making the fourth embodiment of lead body 120 (shown in FIG. 11) will now be described. Each conductor 1020 of a plurality of conductors 1020 is coated with a layer 1030 of extrusion material. Each conductor 1020 of the plurality of conductors 1020 is cylindrically wrapped around (i.e. coiled around) a cylindrically shaped mandrel (not shown). After the lead body 120 is removed from the mandrel, the space formerly occupied by the mandrel will form lumen 1040 between the plurality of conductors 1020. The layer of extrusion material 1030 around each conductor 1020 ensures that the conductors 1020 are uniformly spaced as shown in FIG. 10.

In an alternative embodiment of the method of the present invention, each conductor 1020 of the plurality of conductors 1020 is not coiled around a cylindrically shaped mandrel, but instead is placed lengthwise along the axial length of the mandrel.

The combined portions 1000 of lead body assembly 115 are then covered with heat shrink tubing (not shown) and heat is applied. The heat melts the layers 1030 of extrusion material around the plurality of conductors 1020 and the melted extrusion material flows together to form an integral body. The heat shrink tubing holds and compresses the extrusion material around the conductors 1020 that are located within the extrusion material to create a unitary body lead 1100 as shown in FIG. 11. The conductors 1020 in unitary body lead 1100 may each be centered within the wall 1110 of the unitary body lead 1100. Wall 1110 is formed from the layers that include the layers 1030 of extrusion material shown in FIG. 10. The unitary body lead 1100 is then cooled and the heat shrink tubing removed. Lumen 1120 is formed when the unitary body lead 1100 is removed from the mandrel (not shown). There may be some release of coiled tension in the conductors when the heat shrink tubing is removed.

The lead body assembly 115 shown in FIG. 10 has been described as having cylindrical symmetry. It is noted that other types of geometrical cross-sectional shapes (e.g., rectangular) could be used if a different shape is desired for a particular application.

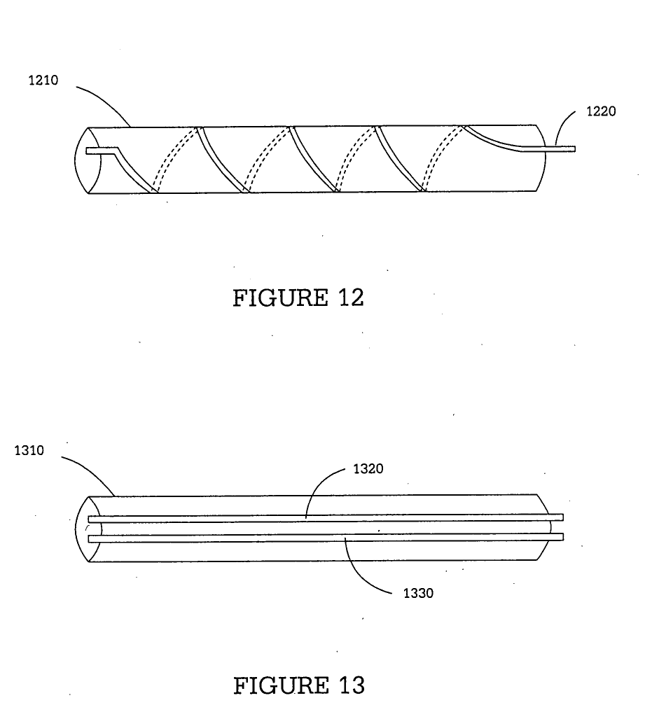

FIG. 12 illustrates a perspective side view of a mandrel 1210 showing how an exemplary conductor 1220 may be coiled around the axial length of the mandrel 1220. Other conductors (not shown in FIG. 12) may also be coiled around mandrel 1210 adjacent to conductor 1220.

FIG. 13 illustrates a perspective side view of a mandrel 1310 showing how a plurality of conductors may be placed along the axial length of the mandrel 1310. Two exemplary conductors, 1320 and 1330, are shown in FIG. 13 placed along the length of mandrel 1310. Other conductors (not shown in FIG. 13) may also be placed along the length of mandrel 1310 adjacent to conductors 1320 and 1330.

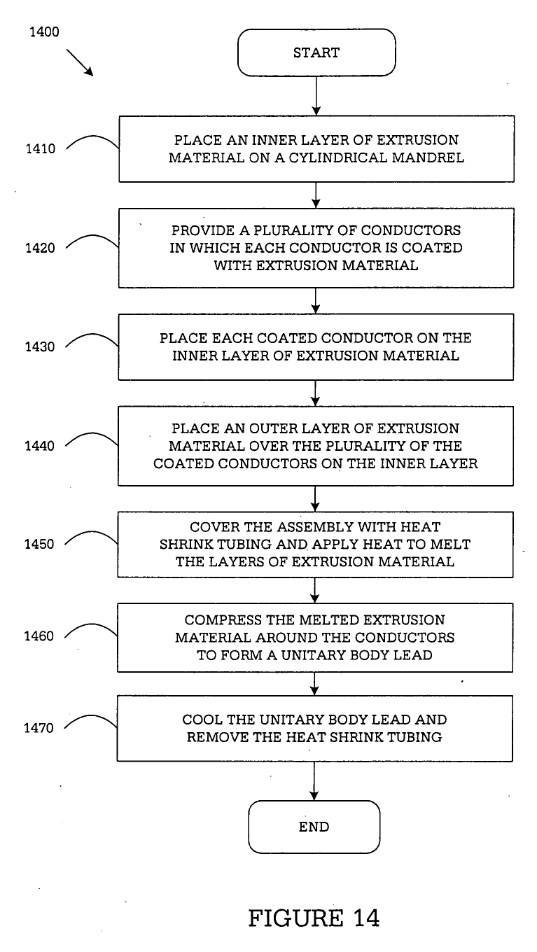

FIG. 14 illustrates a flow chart depicting the steps of one advantageous embodiment of the process of the present invention for making the first embodiment of lead body 120. The steps of the method are collectively referred to with reference numeral 1400.

An inner layer of extrusion material is placed on a cylindrical mandrel (step 1410). A plurality of conductors is provided in which each conductor is coated with extrusion material (step 1420). Each coated conductor is then wrapped around (or placed on) the inner layer of extrusion material (step 1430). An outer layer of extrusion material is then placed over the plurality of coated conductors on the inner layer (step 1440).

The assembly of the inner layer, the coated conductors, and the outer layer is then covered with heat shrink tubing and heat is applied to melt the layers of extrusion material (step 1450). The heat shrink tubing compresses the extrusion material around the conductors to form a unitary body lead (step 1460). The unitary body lead is then cooled and the heat shrink tubing is removed (step 1470).

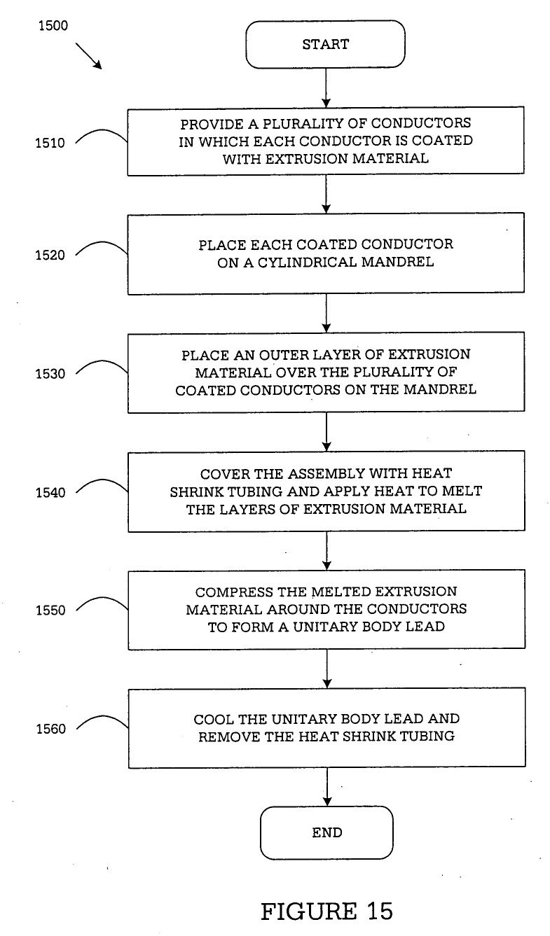

FIG. 15 illustrates a flow chart depicting the steps of an advantageous embodiment of the method of the present invention for making the second embodiment of lead body 120. The steps of the method are collectively referred to with reference numeral 1500.

A plurality of conductors is provided in which each conductor is coated with extrusion material (step 1510). Each coated conductor is then wrapped around (or placed on) a cylindrical mandrel (step 1520). An outer layer of extrusion material is then placed over the plurality of coated conductors on the cylindrical mandrel (step 1530).

The assembly of the coated conductors and the outer layer is then covered with heat shrink tubing and heat is applied to melt the layers of extrusion material (step 1540). The heat shrink tubing compresses the extrusion material around the conductors to form a unitary body lead (step 1550). The unitary body lead is then cooled and the heat shrink tubing is removed (step 1560).

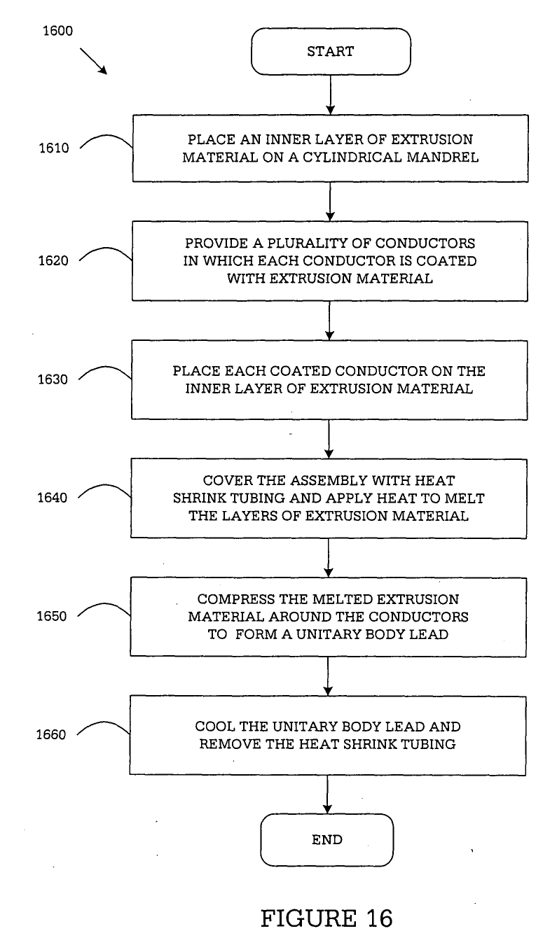

FIG. 16 illustrates a flow chart depicting the steps of an advantageous embodiment of the method of the present invention for making the third embodiment of lead body 120. The steps of the method are collectively referred to with reference numeral 1600.

An inner layer of extrusion material is placed on a cylindrical mandrel (step 1610). A plurality of conductors is provided in which each conductor is coated with extrusion material (step 1620). Each coated conductor is then wrapped around (or placed on) the inner layer of extrusion material (step 1630).

The assembly of the inner layer and the coated conductors is then covered with heat shrink tubing and heat is applied to melt the layers of extrusion material (step 1640). The heat shrink tubing compresses the extrusion material around the conductors to form a unitary body lead (step 1650). The unitary body lead is then cooled and the heat shrink tubing is removed (step 1660).

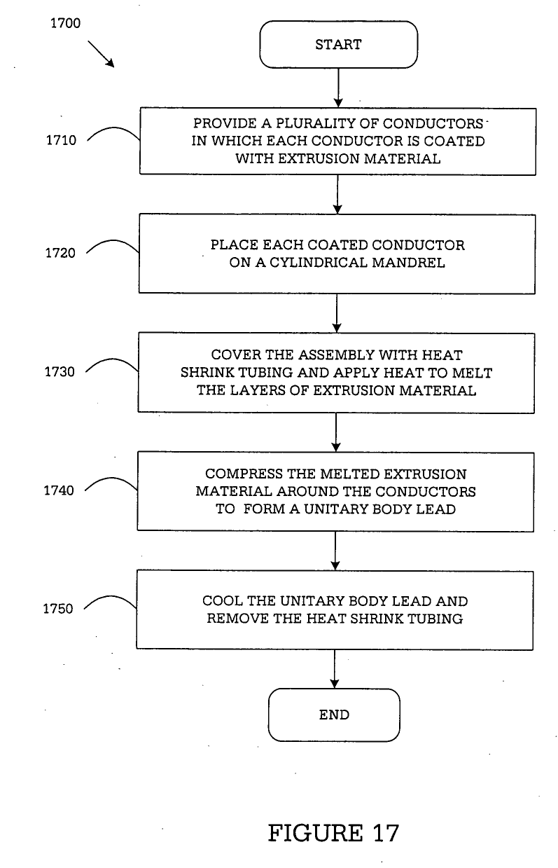

FIG. 17 illustrates a flow chart depicting the steps of an advantageous embodiment of the method of the present invention for making the fourth embodiment of lead body 120. The steps of the method are collectively referred to with reference numeral 1700.

A plurality of conductors is provided in which each conductor is coated with extrusion material (step 1710). Each coated conductor is then wrapped around (or placed on) a cylindrical mandrel (step 1720). The assembly of the coated conductors is then covered with heat shrink tubing and heat is applied to melt the layers of extrusion material (step 17930). The heat shrink tubing compresses the extrusion material around the conductors to form a unitary body lead (step 1740). The unitary body lead is then cooled and the heat shrink tubing is removed (step 1750).

It may be advantageous to set forth definitions of certain words and phrases that may be used within this patent document: the terms “include” and “include,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

1. A method of manufacturing a lead comprising the steps of:
placing an inner layer of extrusion material on a mandrel;
placing at least one conductor coated with a layer of extrusion material on the inner layer of extrusion material;
placing an outer layer of extrusion material over the at least one conductor coated with a layer of extrusion material to form a lead body assembly;
forming the lead body assembly, wherein the formed lead body assembly contains a unitary wall and wherein the conductors are within the unitary wall;
attaching at least one electrode to the at least one conductor at a distal end of the lead body; and
attaching at least one connector to the at least one conductor at a proximal end of the lead body.

2. The method as claimed in claim 1 wherein the forming step further comprises the steps of:
placing heat shrink tubing over the lead body assembly;
heating the lead body assembly to melt the extrusion material in the lead body assembly;
compressing the melted extrusion material around the at least one conductor coated with a layer of extrusion material in the lead body assembly;
cooling the lead body assembly to form the lead body; and
removing the heat shrink tubing from the lead body.

3. The method as claimed in claim 1 wherein the extrusion material of the inner layer and the extrusion material on the at least one conductor coated with a layer of extrusion material and the extrusion material of the outer layer are formed from the same type of extrusion material.

4. A method of manufacturing a lead body comprising the steps of:
placing at least one conductor coated with a layer of extrusion material on a mandrel; and
placing an outer layer of extrusion material over the at least one conductor coated with a layer of extrusion material to form a lead body assembly.

5. The method as claimed in claim 4 further comprising the steps of:
placing heat shrink tubing over the lead body assembly;
heating the lead body assembly to melt the extrusion material in the lead body assembly;
compressing the melted extrusion material around the at least one conductor coated with a layer of extrusion material in the lead body assembly;
cooling the lead body assembly to form the lead body; and
removing the heat shrink tubing from the lead body.

6. The method as claimed in claim 4 wherein the extrusion material on the at least one conductor coated with a layer of extrusion material and the extrusion material of the outer layer are formed from the same type of extrusion material.

7. A method of manufacturing a lead body comprising the steps of:
placing an inner layer of extrusion material on a mandrel; and
placing at least one conductor coated with a layer of extrusion material on the inner layer of the extrusion material on the mandrel.

8. The method as claimed in claim 7 further comprising the steps of:
placing heat shrink tubing over the lead body assembly;
heating the lead body assembly to melt the extrusion material in the lead body assembly;
compressing the melted extrusion material around the at least one conductor coated with a layer of extrusion material in the lead body assembly;
cooling the lead body assembly to form the lead body; and
removing the heat shrink tubing from the lead body.

9. The method as claimed in claim 7 wherein the extrusion material on the at least one conductor coated with a layer of extrusion material and the extrusion material of the inner layer are formed from the same type of extrusion material.

10. A method of manufacturing a lead body comprising the steps of:
providing at least one conductor coated with a layer of extrusion material; and
placing the at least one conductor coated with a layer of extrusion material on a mandrel to form a lead body assembly.

11. The method as claimed in claim 10 further comprising the steps of:
placing heat shrink tubing over the lead body assembly;
heating the lead body assembly to melt the extrusion material in the lead body assembly;
compressing the melted extrusion material around the at least one conductor coated with a layer of extrusion material in the lead body assembly;
cooling the lead body assembly to form the lead body; and
removing the heat shrink tubing from the lead body.

12. A lead for implantation in a human body, the lead comprising:
a lead body comprising of:
a unitary wall having an inner portion that forms a lumen; and
one or more conductors wherein the one or more conductors are spirally wound around the lumen and are within the unitary wall;
at least one electrode located at a distal end of the lead body; and
at least one connector located at a proximal end of the lead body, wherein the at least one connector and at least one electrode are connected by at least one conductor.

13. The lead as claimed in claim 12 wherein the unitary wall is comprised of extrusion material.

14. The lead as claimed in claim 12 wherein no electrical insulation material is between the conductors and the unitary wall.

15. The lead as claimed in claim 12 further wherein the diameter of the lead is no greater than 34 French.

16. The lead as claimed in claim 15 further comprising of at least five electrodes.

17. A system for stimulating a portion of a human body, wherein the system comprises:
a source for generating a stimulus; and
a lead connectable to the source for receiving the stimulus from the source, wherein the lead comprises:
a lead body comprising:
a unitary wall having an inner portion that forms a lumen; and
at least one conductor wound around the lumen and within the unitary wall;
at least one electrode located at a distal end of the lead body; and
at least one connector located at a proximal end of the lead body, wherein the at least one connector and the at least one electrode are connected by the at least one conductor.

18. The system as claimed in claim 17, wherein the unitary wall is comprised of extrusion material.

19. The system as claimed in claim 17 wherein no electrical insulation material is between the conductors and the unitary wall.

20. The system as claimed in claim 17 wherein the diameter of the lead is no greater than 34 French.

21. The system as claimed in claim 15 wherein the lead comprises at least five electrodes.

22. A method of manufacturing a lead for stimulation comprising the steps of:
placing at least one conductor coated with a layer of extrusion material on a mandrel;
forming a unitary lead body assembly with a lumen and with at least one conductor within a unitary wall and spirally wound around the lumen;
attaching at least one electrode to the at least one conductor at a distal end of the lead body; and
attaching at least one connector to the at least one conductor at a proximal end of the body.

23. The method as claimed in claim 22 wherein the step of placing further comprises the steps of placing a first layer of extrusion material on the mandrel.

24. The method as claimed in claim 22 wherein the step of forming further comprises the steps of:
placing heat shrink tubing over the lead body assembly;
heating the lead body assembly to melt the extrusion material to form a unitary body; and
removing the heat shrink tubing from the lead body.

25. The method as claimed in claim 24 further comprising the step of compressing the melted extrusion material around the at least one conductor coated with a layer of extrusion material in the lead body assembly.

26. The method as claimed in claim 23 wherein the extrusion material is placed exterior to the at least one conductor.

27. The method as claimed in claim 23 wherein the extrusion material is placed interior to the at least one conductor.

28. The method as claimed in claim 27 wherein a second layer of extrusion material is placed exterior to the at least one conductor.

29. A method of manufacturing a lead body comprising the steps of:
placing an inner layer of extrusion material on a mandrel;
placing at least one conductor coated with a layer of extrusion material on the inner layer of extrusion material placed on the mandrel; and
placing an outer layer of extrusion material over the at least one conductor coated with a layer of extrusion material to form a lead body assembly.

30. The method as claimed in claim 29 further comprising the steps of:
placing heat shrink tubing over the lead body assembly;
heating the lead body assembly to melt the extrusion material in the lead body assembly;
compressing the melted extrusion material around the at least one conductor coated with a layer of extrusion material in the lead body assembly;
cooling the lead body assembly to form the lead body; and
removing the heat shrink tubing from the lead body.

31. The method as claimed in claim 29 wherein the extrusion material of the inner layer and the extrusion material on the at least one conductor coated with a layer of extrusion material and the extrusion material of the outer layer are formed from the same type of extrusion material.

32. A lead body assembly comprising:
an inner layer of extrusion material;
a plurality of conductors wherein each conductor of the plurality of conductors is coated with a layer of extrusion material and wherein each conductor of the plurality of conductors is placed on the inner layer of extrusion material; and
an outer layer of extrusion material placed over the plurality of conductors.

33. A lead body assembly as claimed in claim 32 that has been subjected to heat and compression to form a lead body.

34. A lead body assembly comprising:
a plurality of conductors wherein each conductor of the plurality of conductors is coated with a layer of extrusion material; and
an outer layer of extrusion material placed over the plurality of conductors.

35. A lead body assembly as claimed in claim 34 that has been subjected to heat and compression to form a lead body.

36. A lead body assembly comprising:
an inner layer of extrusion material; and
a plurality of conductors wherein each conductor of the plurality of conductors is coated with a layer of extrusion material and wherein each conductor of the plurality of conductors is placed on the inner layer of extrusion material.

37. A lead body assembly as claimed in claim 36 that has been subjected to heat and compression to form a lead body.

38. A lead body assembly comprising:
a plurality of conductors wherein each conductor of the plurality of conductors is coated with a layer of extrusion material.

39. A lead body assembly as claimed in claim 38 that has been subjected to heat and compression to form a lead body.

40. A method of manufacturing a lead body comprising the steps of:
placing at least one conductor coated with a layer of extrusion material on a mandrel; and
forming a lead body assembly that includes the at least one conductor coated with a layer of extrusion material.

41. The method as claimed in claim 40 further comprising the steps of:
placing heat shrink tubing over the lead body assembly;
heating the lead body assembly to melt the extrusion material in the lead body assembly;
compressing the melted extrusion material around the at least one conductor coated with a layer of extrusion material in the lead body assembly;
cooling the lead body assembly to form the lead body; and
removing the heat shrink tubing from the lead body.

42. A lead body comprising a plurality of conductors in which each conductor of the plurality of conductors is coated with a layer of extrusion material.

43. A lead body as claimed in claim 42 further comprising an inner layer of extrusion material placed adjacent to the plurality of conductors that are coated with a layer of extrusion material.

44. A lead body as claimed in claim 43 further comprising an outer layer of extrusion material placed adjacent to the plurality of conductors that are coated with a layer of extrusion material.
